(19) United States
(12) Patent Application Publication
(10) Pub. No.: US 2026/0283569 A1
(43) Pub. Date: Sep. 24, 2026

(54) A PORTABLE AND COMPACT POSITRON EMISSION TOMOGRAPHY (PET) SYSTEM WITH REAL-TIME ADAPTABILITY

(71) Applicants: Mei-Yin Chou, Atlanta, GA (US); ACADEMIA SINICA, Taipei City (TW)

(72) Inventors: MYTHRA VARUN NEMALLAPUDI, New Taipei City (TW); Chih-Hsun Lin, Taipei City (TW); Shih-Chang Lee, Taipei City (TW)

(73) Assignees: Mei-Yin Chou, Atlanta, GA (US); ACADEMIA SINICA, Taipei City, TW (TW)

(21) Appl. No.: 19/477,484

(22) PCT Filed: Apr. 26, 2024
(86) PCT No.: PCT/US2024/026361

§ 371 Date: Oct. 21, 2025

Related U.S. Application Data
(60) Provisional application No. 63/498,530, filed on Apr. 27, 2023.

(51) Int. Cl. A61B 6/03; A61B 6/00; A61B 6/46

(52) U.S. Cl. CPC A61B 6/037; A61B 6/4441; A61B 6/4458; A61B 6/465

(57) ABSTRACT

The invention is related to a portable and compact positron emission tomography (PET) system with real-time adaptability, a detector unit contained therein, and an image reconstruction method. The system comprises at least one detector unit, including at least one compact, handheld gamma detector module for front end readout, for processing signals to output serialized data, at least one connecting cable, and a DAQ unit for data acquisition; a computer, interfaced with the at least one detector unit, a robot unit, a user interface and visualization unit, and a perception unit, for processing the received data to generate appropriate responses for each of the units; a perception unit; and a user interface and visualization module, for displaying reconstructed and co-registered images of a patient to be detected in real time.

### FIELD OF THE INVENTION

The invention is related to a portable and compact positron emission tomography (PET) system with real-time adaptability, or a single photon emission computed tomography (SPECT) imaging system. In particular, the invention is a portable and compact tool for field use not only to diagnose neurodegenerative diseases such as dementia.

### BACKGROUND OF THE INVENTION

Current PET systems are large and expensive and limit the widespread usage and access. Observing targeted regions in a patient, or adapting the diagnosis real-time can be achieved by a system that can be freely manipulated with minimum restrictions to the patient.

### SUMMARY OF THE INVENTION

The invention addresses such issues to propose a compact imaging system to overcome such challenges. Such a compact system needs to handle data, power and heat effectively to work independent of a static framework in which to operate. The imaging system of the invention provides a compact and portable positron emission tomography (PET) system, or a single photon emission computed tomography (SPECT) imaging system, for close range detection and organ specific imaging, majorly including: a robot unit to support, position and orient the detection unit in planned configurations real-time; a perception unit, including a vision module to track the patient movements, and provide guidance for the robot unit to orient the imaging system while achieving motion correction by maintaining a constant relative orientation between the imaging system and the patient to be detected for a given subset of the acquisition; a computer to coordinate the major units and implement the image acquisition in a planned sequence of orientations to image a targeted region of interest; and a user interface and visualization unit to view the patient in real-time, and to visualize the reconstructed PET activity image superimposed on a variation of the patient which can be a computed tomography (CT) image or magnetic resonance imaging (MRI) image. The invention enables a seamless operation with maximum automation; can easily be transported from place to place; can easily be assembled and operated in remote locations; is low cost and widely adoptable.

The invention provides a portable and compact positron emission tomography (PET) system with real-time adaptability, comprising: at least one detector unit, including at least one compact, handheld gamma detector module for front end readout, being provided to convert gamma into visible light and convert the visible light into analog electrical signals, and to process signals for outputting serialized data, at least one connecting cable, interfacing with the at least one detector unit, and a data acquisition unit (DAQ) unit for data acquisition, being used to deserialize the data, and to proceed with configurating settings; a computer, or central processing unit (CPU), interfaced with the at least one detector unit, a robot unit, a user interface and visualization unit, and a perception unit, for issuing control signals, receiving data from each unit, and processing the received data to generate appropriate responses for each of the units; a robot unit, interfaced with the CPU, for mechanically holding, positioning and orienting each of the at least one detector unit, including: a C-arm holder for holding the at least one detector unit, a robotic plural-axis arm, connected to the C-arm holder, for accepting inputs from the computer for a final position, speed and configuration, a height adjust arm for adjusting vertical based on the inputs from the computer height, and a distance adjust arm for horizontally adjusting distance from the at least one detector unit, and a controller, coupled with the computer, for controlling positioning of the robotic plural-axis arm, the height adjust arm and the distance adjust arm to an appropriate place for acquiring images; a perception unit comprising sensors to include a vision unit, for performing calibration using a 1D, 2D or 3D standard reference signal and matching coordinates with the, robotic unit to capture position and orientation of a patent to be detected; and a user interface and visualization module, for a user to input variables for a current imaging plan, and for displaying reconstructed and co-registered images of a patient.

The invention further provides a detector unit for use in a portable and compact positron emission tomography (PET) system, comprising: At least one crystal, for converting gamma emitted from a target region of a patient to be detected and attached with radioactive markers, into visible light in proportion to energy of gamma photon; a silicon photo-multiplier (SiPM) for detecting the visible light from the at least one crystal(s) and converting the visible light into analog electrical signals that are proportional to the input visible light and correlated to a arrival time of events and light intensity, so as to detect gamma ray incident onto the crystal at a crystal level, in which multiple channels are provided on each SiPM, wherein up to 64 channels are connected one-to-one with a reading means for reading, a reading means, for receiving the electrical signals from a corresponding SiPM at a channel level, wherein inside the reading means, the SiPM signal is converted into a time of threshold signal and start and end timestamps are registered by the time to a digital converter, the timestamps represent information of the arrival time, and the approximated energy of the original gamma event, in which the digitized event is stored in a 48 bit event along with all the 64 channels of the reading means in an internal FIFO, and the data from each reading means is sent out serially, an field programmable gate array (FPGA), for reading the data from the reading means, arranging the data from multiple reading means and passes it to a serializer to serially send the data along a connecting cable lines in a loss-free manner; a data acquisition unit (DAQ), including: a de-serializer, for receiving the event data from the corresponding serializer and de-serialize it via a GMSL protocol, an FPGA-DAQ, for receiving the de-serialized event data from each separate de-serializer and arranging the same in an internal FIFO to arrive at an unbiased acquisition from multiple data streams, in which packaged information is then sent out to the computer via a bridge chip, and the data is received by a computer and saved; a connecting cable, for interfacing with the DAQ unit, which is a USB3.0 connector that is a compatible cable, wherein the connecting cable has two channels of serial link, two sets of I/O pins for configuration and control, two RST channels, two clock channels, and two channels for power; and a computer, interfaced with the other major units of the signal and data processing system by issuing control signals, receiving the data from each unit, and processing the received data to generate appropriate responses, for each of the major units, allowing the signal and data processing system to function as a state machine, which interacts and generates responses based on the configuration of the patient, wherein for every short interval of time, the computer inputs information from a perception unit, compares with an imaging plan, calculates a target position of the robotic arm, issues commands to a robotic unit, performs image reconstruction and co-registration with a pre-existing DICOM (CT or MRI) image of the patient, or another closely matched DICOM image, and sends the current registered image to a visualization unit for real-time inspection.

The invention still further provides a multi-angular reconstruction method for use in a PET system, involving a number of stages for forward and back projections on each separate data corresponding to a different angle, relating to a detecting area, in which a base space is defined with coordinate axes, wherein each rotated orientation recovers an image from a previous angle that is reoriented to a current angle and reconstructed, eventually coming back to the base space, and an iterative reconstruction is performed in accordance to an algorithm as follows:

$${f}_{i}^{k + 1 , {θ}_{L - 1}} = \frac{{R}_{{θ}_{L}}^{- 1} \text{?} \{ {f}_{i}^{k + 1 - α , {θ}_{L}} \text{?} \}}{{\sum }_{jLOR} \text{?} {H}_{ij}} \times {\sum }_{jLOR} \text{?} \frac{{g}_{j}^{{θ}_{L - 1}} {H}_{ij}}{{\sum }_{{i}^{′} pixels} {R}_{θ}^{- 1} \text{?} \{ {f}_{{i}^{′}}^{k + 1 - α , {θ}_{L -}} \text{?} \} {H}_{{i}^{′} j}}$$

$$\text{?} \text{indicates text missing or illegible when filed}$$

where,
i represents a pixel number,
j represents a line of response (LOR),
k represents an iteration number from 1 to N,
L is a total number of rotation angles θ₁ . . . θ_{L},
1 represents the angular sub-iteration ranging from 0 to L−1,
α is defined such that
α=1 for 1=0 and
α=0 for 1>0,

$${R}_{{θ}_{a} , {θ}_{b}}^{- 1}$$

represents the rotation of the image from θₐ to θ_{b},
   wherein for each iteration of an MLEM reconstruction, all L angular sub-iterations are performed and during each sub-iteration, an image from a previous step is rotated into a current sub-iteration and the rotated image is forward projected, and divided by the measured data for that angle, and then back projected, which result is multiplied by the ith pixel of the rotated image from the previous sub-iteration and normalized by a system response matrix.

### BRIEF DESCRIPTION OF THE DRAWINGS

FIG. 1, including FIG. 1(a), FIG. 1(b), and FIG. 1(c), respectively shows a schematic diagram of the complete PET imaging system according to the invention, a physical model of the overall structure of the PET system according to the invention, and a block diagram of the robotic unit contained in the PET system of the invention.

FIG. 2 is a block diagram showing operations of the detector unit 110, including at least one compact, handheld gamma detector module, of the invention.

FIG. 3 is a block diagram illustrating operations of multiple detector units according to the invention.

FIG. 4 shows a simplified signal flow diagram of a detector unit according to the invention.

FIG. 5 shows a block diagram of the perception unit describing the subcomponents of depth sensing camera, proximity sensors for safety, and the temperature sensor for ambient temperature monitoring.

FIG. 6 shows a block diagram of the user interface and visualization unit describing the subcomponents of the system.

FIG. 7 shows channels threshold corrected, and calibrated for absolute Egamma, in which on the left is shown a sample of 16 channels energy spectrum for a standard radioactive 22Na source and for the intrinsic activity from the crystal, and on the right is shown the energy spectrum for a standard 22Na source for all the 1024 channels after they have been calibrated for precise energy prediction, wherein an energy resolution of 8% Full Width at Half Maximum (FWHM) was achieved.

FIG. 8 shows time resolution measured as the FWHM of the time delay histogram between a reference channel in opposition to all the 512 channels of a module showing a coincidence time resolution of 282 ps FWHM.

FIG. 9 shows a breakdown of the major units of the detector unit included in the PET system of the invention.

### DETAILED DESCRIPTION OF THE INVENTION

As provided in the drawings, the portable and compact positron emission tomography (PET) system with real-time adaptability according to the invention essentially comprises a robot unit, at least one detector unit for processing PET signals and data, and a reconstruction method.

The at least one detector unit for processing PET signals and data according to the invention comprises the following: at least one compact, handheld gamma Detector module: Front End Readout: Power and HV circuits: HV circuit: Circuitry to supply the requisite voltage to the SiPM, accepting an input voltage, and inputs on the HV set value and enable from the FPGA. HV DAC: Circuitry to accept input from the FPGA and set a unique HV value to the designated HV circuit. Temperature sensor: Reads the temperature value and sends the digital output to the FPGA. Regulator 15->1.8V: Circuitry to accept an input voltage in the range of 5-15V and output a voltage of 1.8 V to power the on-board electronics. Regulator 15->3.3V: Circuitry to accept an input voltage in the range of 5-15V and output a voltage of 1.8 V to power the on-board electronics. Clock circuit: Programmable clock generator to accept input from a crystal oscillator and generate multiple clock outputs that are routed through the clock buffer to multiple reading means, or ASICs. The clocks are input to the ASIC for serial data streaming and to provide a reference for the PLL within the ASIC. SiPM: Detects visible light from inorganic scintillating crystals and converts them into electrical signals that are correlated to the arrival time of the events and the light intensity. The object is to detect an incoming gamma photon into the PET module at a crystal level. There are multiple channels on each SiPM, up to 64 are connected one-to-one with the readout ASIC. Crystal: This is any inorganic scintillator that can convert gamma into visible light. This component is optically coupled to SiPM sensitive side to ensure maximum light transfer. The coupling between SiPM and crystal can be one to one, or many to one, or one to many, or many to many. Light sharing and isolation if used can be achieved by a variety of techniques such as, usage of fully reflective materials between each crystal, usage of partially reflective materials between two materials, usage of another light sharing materials on any of the crystal surfaces that ensures the transfer of visible light from one crystal to another. The reflective materials can be specular reflectors, or diffusive reflectors, or partial reflectors. ASIC: The ASIC reads the signals from a SiPM at a channel level, shapes the signal, discriminates the signal for pre-set threshold values, and encodes the arrival time and the time over threshold by employing a tuned voltage controlled oscillator and a Time to Digital Converter. The events are stored in internal buffers and transmitted out serially. The ASIC can be configured to adjust the threshold, mask individual readout channels, tune the PLL, and adjust the analog circuit settings. The ASIC used in this circuit is STIC3 developed in Heidelberg. However, any circuit capable of reading out SiPM signals, and extracting the arrival time in the range of 1 picosecond to 10 nanoseconds, and extracting the energy information, packing them into individual events several bytes long is usable for the purpose of this system design. Up-to 64 channels of SiPM are read one-to-one by the ASIC. Field programmable gate array (FPGA)-Front end: The central element of the front end module is an FPGA-front end that is used to configure the ASIC, adjust the HV circuits, read the temperature data from the Temperature sensor, read the serial data from multiple ASICS and arrange them into a data stream, and communicate with the DAQ through a connection that is used to accept external configuration commands, and to transmit the digitized event data from the ASIC. Each FPGA is connected to multiple ASICs for control and data acquisition. Serializer circuit: This is used to serialize the data from the FPGA following a serial protocol and transmitting them towards the DAQ. Each serializer circuit is dedicated per FPGA. Module:Connecting cable: A USB3.0 connector compatible cable rated to operate >3 Amps at >15V is used to interface the front-end board with the DAQ board. The cable has two channels of serializer, two sets of I/O pins for configuration and control, two RST channels, two clock channels, and two channels for power. DAQ board: de-serializer: Receives event data from the front end board via serializing protocol, and converts the data back into the original bit stream of sequential events of fixed size. This data is sent to the FPGA-DAQ. FPGA-DAQ: The central element of the DAQ board is the FPGA-DAQ which receives the deserialized event data from multiple serializer inputs, arranges them into separate FIFO buffers, ensuring a uniform acquisition from all the external readout sources, issues RST signals to the ASIC upon requests from the user software arriving through the USB connection, accept user requests and configuration settings for the front end board including ASIC configuration, high voltage settings, clock settings, configuring the acquisition of the FPGA-front end and forwarding the configurable settings via a dedicated Input/Output line on the Connecting cable (USB3.0). Power circuits:Power: Accepts a 5V-15V and 0-6A external input from a dedicated DC power supply. This sends dedicated power lines through the Connecting cable to power the regulators, and HV circuits on the front end board. Regulator 15->3.3V: Circuitry to accept an input voltage in the range of 5-15V and output a voltage of 1.8 V to power the on-board electronics. FTDI bridge chip: This circuit forms the interface between the FPGA-DAQ and the external computer or a Communication hub that hosts the user interface software. It is used to communicate the configuration settings issued by the host, and to transmit the received events from the FPGA-DAQ towards the host computer. Communication hub: This is an electronics system that interacts with multiple Detector sets and connects them to a single Computer (or Central Processing Unit).

### Computer (or CPU, CENTRAL PROCESSING UNIT):

The CPU interfaces with all the other major units of the system by issuing control signals, receiving the data from each unit, and processing the received data to generate the appropriate responses, for each of the major units. The CPU also contains several instances of pre-loaded imaging plans which allow the system to function as a state machine which interacts and generates responses based on the configuration of the human patient. For every small interval of time, the CPU inputs information from the Perception Unit, compares with the imaging plan, and calculates the target position of the robotic arm, and issues the commands to the Robotic unit. The CPU performs the image reconstruction and co-registration with a pre-existing CT image of the patient, or another closely matched DICOM image. The CPU sends the current registered image to the visualization system for real-time inspection.

### Reconstruction Techniques:

### Multi-Angular Reconstruction:

Multi-angle reconstruction involves subsequent stages for forward and back projections on each separate data corresponding to a different angle. A base space is defined with the coordinate axes as shown in the figure of the full system.

Each rotated orientation recovers an image from the previous angle that is reoriented to the current angle and reconstructed, eventually coming back to the base space. The iterative reconstruction is performed in accordance to the algorithm shown below.

$${f}_{i}^{k + 1 , {θ}_{L - 1}} = \frac{{R}_{{θ}_{L}}^{- 1} \text{?} \{ {f}_{i}^{k + 1 - α , {θ}_{L}} \text{?} \}}{{\sum }_{jLOR} \text{?} {H}_{ij}} \times {\sum }_{jLOR} \text{?} \frac{{g}_{j}^{{θ}_{L - 1}} {H}_{ij}}{{\sum }_{{i}^{′} pixels} {R}_{θ}^{- 1} \text{?} \{ {f}_{{i}^{′}}^{k + 1 - α , {θ}_{L -}} \text{?} \} {H}_{{i}^{′} j}}$$

$$\text{?} \text{indicates text missing or illegible when filed}$$

where,
i represents the pixel number
j represents the Line of Response (LOR)
k represents the iteration number from 1 to N
L is the total number of rotation angles θ₁ . . . θ_{L}
1 represents the angular sub-iteration ranging from 0 to L−1
α is defined such that
α=1 for 1=0
α=0 for 1>0

$${R}_{{θ}_{a} , {θ}_{b}}^{- 1}$$

represents the rotation of the image from θₐ to θ_{b}.

For each iteration of the MLEM reconstruction, all the L angular sub-iterations are performed.

During each sub-iteration, the image from the previous step is rotated into the current sub-iteration and this rotated image is forward projected, and divided by the measured data for that angle, and then back projected. This result is multiplied by the ith pixel of the rotated image from the previous sub-iteration and normalized by the system response matrix.

### Update Limited Angle Reconstruction:

The compact system described in this invention has a limited field of view. The technique of reconstruction developed in this invention allows the acquisition of a larger view. The detection unit is rotated around the patient head to three separate angles offset by 120 degrees in order to cover the extent of the brain. The detection system will then be offset in the direction of the rotation axis, to acquire data from a different region. By performing three rotations and two axial positions, we can image the full extent of the brain. Combining different combinations of rotations, and slices, we can acquire the images for various scenarios.

### The Need for Detector Motion:

For a limited Field of View system, the following maneuvers are performed Rotation of the detection system about y axis to cover a larger field of view. Axial motion of the detection system along y axis to cover a larger field of view. Adjustment of the distance between the detection modules along the x axis to address a different biological region of interest.

### Approach on Reconstruction:

Reconstruction techniques that involve subsequent stages for forward and back projections on each separate data corresponding to a different angle. A base space is defined with the coordinate axes as shown in the figure of the full system. Each rotated orientation recovers an image that is reoriented to the base space and iteratively reconstructed.

$${f}_{i}^{n + 1} = {f}_{i}^{n} {R}_{{theta}_{k}}^{- 1} \{ \frac{{R}_{{theta}_{k}} \{ {f}_{ij}^{n} \} \}}{\sum {H}_{ij}} \sum \frac{{g}_{{θ}_{k}}}{\sum {H}_{ij} {R}_{{θ}_{k}} \{ {f}_{ij} \}} \}$$

### Perception Unit:

Depth sensors: Comprises a vision subsystem with time of flight capability to generate a 3D point cloud of the space with objects. The primary purpose is to capture the position and orientation of the patient subject. Proximity sensors: These are infrared emitter-detector modules which generate a corresponding signal in response to the distance from the object to the sensor. Temperature sensor: These record the ambient temperature at various positions around the imaging system. The output of this system will provide a go ahead for the detector system start up. The output of this system will also be useful to update the settings of the detection system. DAQ perception: The data acquisition for perception subsystem receives the data from various components, processes and arranges the data and transmits them to the CPU. It also receives calibration commands from the CPU to calibrate the sensors within the Perception Unit.

USER INTERFACE AND VISUALIZATION UNIT: Terminal: This is the part where the user inputs the variables for the current imaging plan. Viewer: Viewer is a screen for monitoring the patient 3D image as viewed by the Perception unit. The viewer consists of markings which help guide the patient to stay in the most desirable position and orientation. Visualization: This module displays the reconstructed and co-registered image of the patient in real time.

### Robot Unit:

Robotic six axes arm: This is a six axes robotic arm that holds the detector modules with a connecting C-arm. It accepts inputs from the CPU for the final position, speed and configuration. This is connected to the end of the Distance adjustment arm. Height adjustment arm: This is a single axis stage with an adjustable vertical height based on the inputs from the CPU. This arm is placed on the ground, or a table with an immovable base. This arm connects to the Distance adjustment arm. Distance adjustment arm: This is a single axis stage with an adjustable horizontal arm based on the inputs from the CPU. This arm is connected to the top-most part of the Height adjust arm. The end of this arm is used to connect the Robotic six axes arm.

The invention is further described as follows.

DESCRIPTION OF THE COMPACTNESS OF THE SYSTEM: The detector system design presented for the ‘Detection system’ allows the resulting system to be compact and modular. The compactness arises due to the digitization of information early in the data flow which is made possible by our design choices in the detection system. Using the described design, a 512 channel module was developed with a compact form factor of 104 mm×52 mm×60 mm. Within this cuboid structure are present 512 channels of inorganic scintillating crystals, SiPM sensors, two electronics boards for the front end electronics to interact with 512 channels of sensitive detectors, and channel the information out across a USB cable towards the DAQ module. In order to maintain a compact form factor the sensitive area of the detector must occupy a maximum percentage of the overall area of the module in the sensitive field of view. The design ensures that a high signal fidelity is maintained while making the detector compact.

To achieve this:
1. The sensors and the corresponding readout electronic circuits are placed on two sides of the same board. This keeps the area to a minimum possible value.
2. The area occupied by the readout electronics for a given number of channels (512 in the implemented case) is less than the area occupied by the sensors.
3. Heat is dissipated with a minimum increment in the overall area of the detector in the sensitive field of view.
4. SiPMs are thermally isolated from the readout circuit which generates heat.
5. Switching regulators are chosen on the front end module to minimize the heat dissipation, as against linear regulators.
6. Shorter leads from the SiPM (sensor) channels to the inputs of the readout ASIC-ensure a lower capacitance and a higher signal quality.
7. Providing dedicated power circuitry for each ASIC, and each sensor array ensures that the readout channels achieve a maximum isolation.
8. All mechanical parts are designed to be connected to the thermally conductive frame which is the central mechanical structure for a module.

DESCRIPTION OF THE MECHANICAL AND THERMAL DESIGN: The mechanics, including the robot unit, have been designed to support the sensitive detectors with heavy LYSO crystals, the readout electronics boards, and the thermal system which manages the heat dissipation. The power generation of this system is 45 Watts, and has an original design for dissipating the heat generated within the system and to maintain the system at a temperature stable to within 1 C. An Aluminum plate was fabricated to fit between the tight space of the two electronic boards and is thermally coupled to the ASIC by a thermal paste. This plate is screwed on to the external mechanical plate and also coupled by a thermal paste. The external mechanical plate has protruding flat arms in the direction normal to the plane of the sensitive detectors and the readout electronics, and away from the detector. Custom designed heat sinks are then coupled to the external mechanical plate via thermal paste and mechanically secured by screws. A backplane with an electric fan directs the air into the heat sinks to dissipate the heat that originated from the ASIC and was directed to the heat sinks. This design allows us to retain the compactness, and allows the system to be scaled without losing the sensitive area.

With the invention, it provides the following features:

Compactness and portability, maximizing the ratio of sensitive to total area in the FOV, electronics design choices making it compact, thermal design maintaining a high fraction of sensitive area, and removing the need to have external cooling system. This makes it compact and portable. Mechanical design that combines a central mechanical frame serving both mechanically and thermal purposes to ensure compactness and portability. R&Perception claims Corrects for motion blur—Helps with co-registration without need for another CT hardware. Keeps a patient free of constraints to the patient couch, or other restraints. Enables image acquisition in many unusual angles.

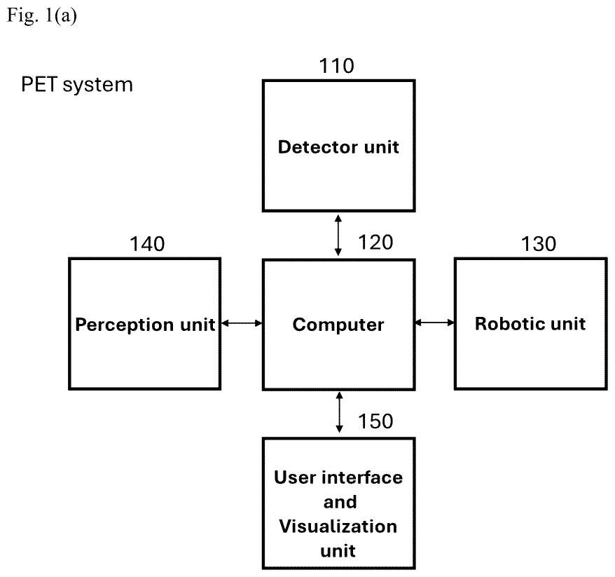

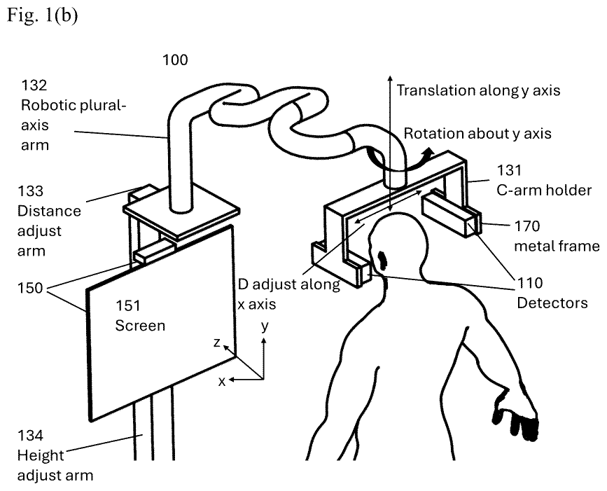

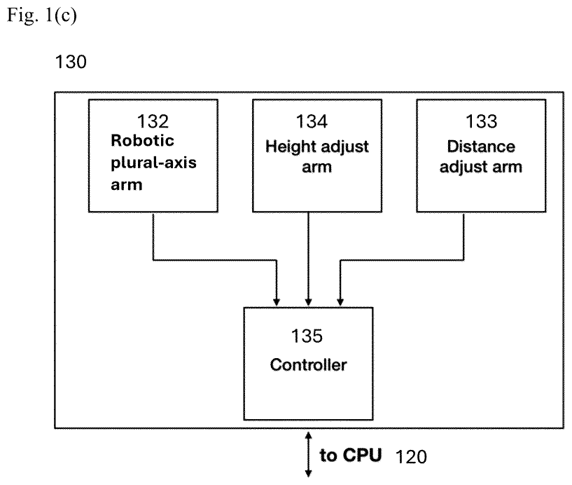

As shown in FIGS. 1(a), 1(b) and 1(c), a portable and compact positron emission tomography (PET) system with real-time adaptability 100 according to the invention comprises:
at least one detector unit 110, including at least one detector module 1101 for front end readout, being a compact, handheld gamma detector module provided to convert gamma into visible light and convert the visible light into analog electrical signals, and to process signals for outputting serialized data, at least one connecting cable 1102, interfacing with the at least one detector unit, and a DAQ unit 1103 for data acquisition, being used to deserialize the data, and to proceed with configurating settings;
a computer 120, interfaced with the at least one detector unit, a robot unit 130, a user interface and visualization unit 140, and a perception unit 150, for issuing control signals, receiving data from each unit, and processing the received data to generate appropriate responses for each of the units;
a robot unit 130, interfaced with the CPU, for mechanically holding, positioning and orienting each of the at least one detector unit, including: a C-arm holder 131 for holding the at least one detector unit 110, a robotic plural-axis arm 132, connected to the C-arm holder 131, for accepting inputs from the computer for a final position, speed and configuration, a height adjust arm 134 for adjusting vertical based on the inputs from the computer height, and a distance adjust arm 133 for horizontally adjusting distance from the at least one detector unit 110 and a detector center and two angular moving stages of the plural-axis arm 132, and a controller 135, coupled with the computer, for controlling positioning of the robotic plural-axis arm 132, the height adjust arm 134 and the distance adjust arm 133 to an appropriate place for acquiring images;
a perception unit 140 comprising sensors, for performing calibration using a 1D, 2D or 3D standard reference signal and matching coordinates with the robotic unit to capture position and orientation of a patent to be detected; and
a user interface and visualization module 150, including a viewer screen 151, for a user to input variables for a current imaging plan, and for displaying reconstructed and co-registered images of a patient to be detected in real time.

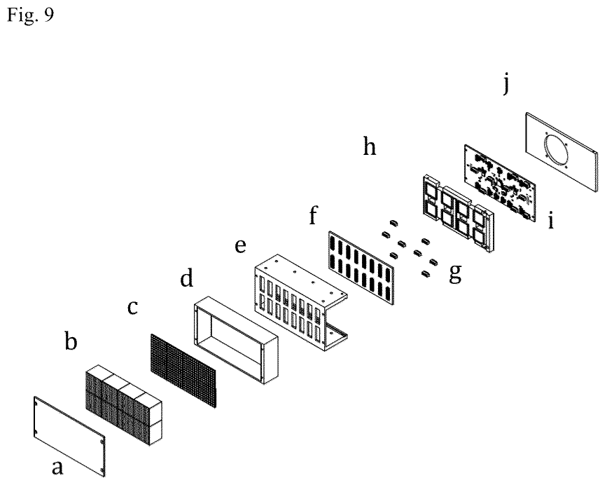

In the robot unit 130 of the portable and compact positron emission tomography (PET) system with real-time adaptability according to the invention, wherein the robotic plural-axis arm is to configure rotating about a y axis, translating along the y axis, and adjusting distance along an X axis, and wherein the robotic plural-axis arm 132 is a six axes arm or a combination of two angular moving stages. Referring back to FIG. 1, the robot unit 130 further comprises a mechanical frame 170 to connect readout boards from at least one detector module 110, for providing conductive path for heat dissipation, and being provided to mount subcomponents contained in the at least one detector unit, sequentially including crystals, SiPM, PCB to contain ASIC, FPGA Front end, serializer and DAQ board, and unit further comprises a chair (not shown) with a neck support to be seated by the patient, while FIG. 9 shows a breakdown of the major units of the detector unit included in the PET system.

In the portable and compact positron emission tomography (PET) system with real-time adaptability of the invention, the perception unit 140 is to track patient movements, and to provide guidance for the robotic plural axis arm to orient the at least one detector unit 110 while achieving motion correction by maintaining a constant relative orientation between the visualization module and the patient for a given subset of the acquisition.

In the PET system 100 of the invention, the user interface and visualization unit 150 further comprises a screen 151 for viewing.

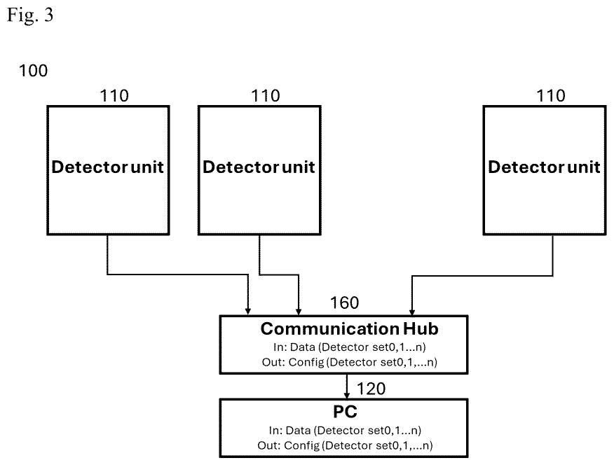

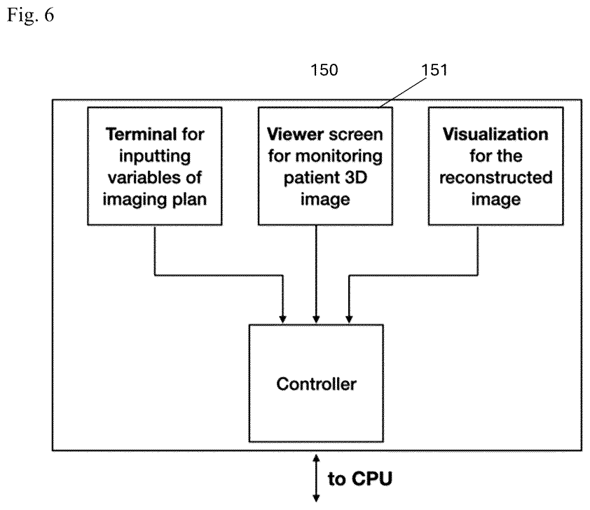

As shown in FIG. 3, in the portable and compact positron emission tomography (PET) system with real-time adaptability of the invention, a communication hub 160 is further provided for interfacing with a plurality of detector units, when being used, and the computer 120. In addition, as shown in FIG. 6 the perception unit further comprises a depth sensing camera, proximity sensors for safety, and a temperature sensor for ambient temperature monitoring.

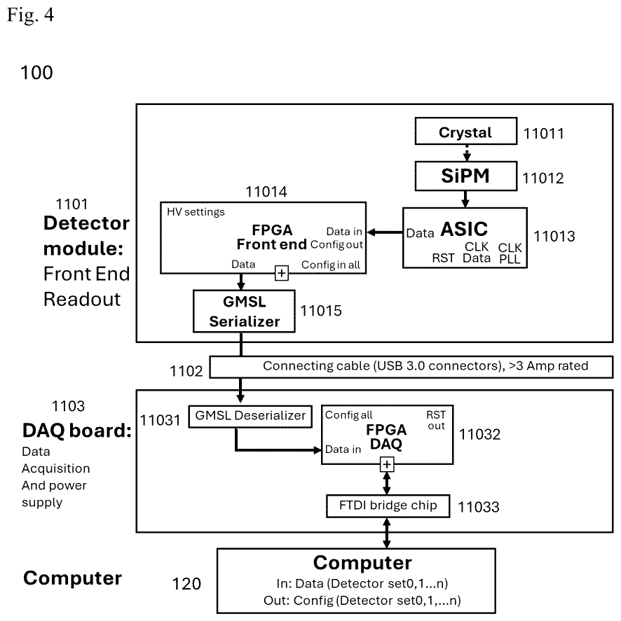

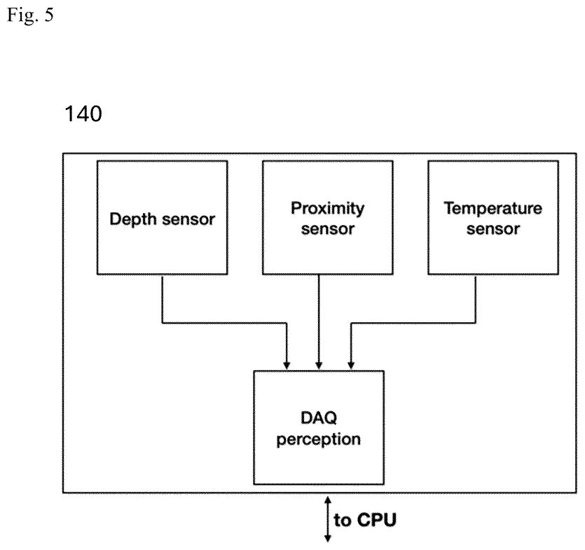

As shown in FIGS. 4 and 5, the compact, handheld gamma detector module 1101 for a positron emission tomography (PET) 100 or single photon emission computed tomography (SPECT) imaging system, comprising: a plurality of scintillating crystals 11011 to detect and convert gamma photons emitted by a patient to be detected to visible light; a plurality of photodetectors 11012 to convert the visible light to analog or digital electric signals, wherein the photodetectors are silicon photo-multipliers (SiPMs); reading means 11013 for reading the analog or digital signals from each of the photodetectors to capture time of arrival and energy of detected gamma event, wherein the reading means are application specific ICs (ASICs); a configurable integrated circuit 11014 for configuring and reading data from the reading means, wherein the configurable integrated circuit may be a field programmable gate array (FPGA) Front end; a data serializer, for serializing the configured data, wherein the data serializer may be a serializer; a plurality of printed circuit boards (PCBs), as shown in FIGS. 10(a) and 10(b), and Table 1, being provided to house electronic components so as to minimize a total volume of space enclosed by the PCBs; and holding means 170, as shown in FIG. 1(b), for mechanically holding the components, which also acts as a means of dissipating heat within the detector module, and thereby to maximize sensitive area while minimizing a total detector module volume, thereby to be unconstrained by external static structure for mechanical, power, thermal or signal flow requirements, and with minimum cabling to ease detector movement.

In the detector module 1101 of the invention, the holding means is a heat sink or a fan.

In the detector module of the invention, wherein the scintillating crystals 11011 are coupled to a single or a plurality of photodetectors 11012, which SiPM arrays, in one to one, one to many or many to one configuration maximizing light transfer.

According to the detector module, each pixel of the SiPM 11013 array is connected one-to-one or many to one to the input channels of the reading means for reading photodetector signals, which is an ASIC with timing resolution value ranging from 1 picosecond to 10 nanoseconds, that digitizes the time and energy information of each gamma event and streams out the data generated in all its input channels via a high-speed interface.

In the detector module of the invention, the configurable integrated circuit 11014 is a FPGA that configures and reads data from one or several ASICs, configures the photodetectors, reads temperature sensors, and sends data to a data acquisition (DAQ) unit, wherein the gamma detecting crystals 11011 glued to the photodetector device 11012 are connected via board-to-board connectors to one side of a PCB, which contains on the other side the reading means 11013 for reading the photodetector and board-to-board connectors, where a distance from the photodetector to the reading means for reading the photodetector, to which it is connected is minimized; wherein the configurable integrated circuit 11014 is soldered to one side of a PCB, which contains on the other side board-to-board connectors and other non-high-power-dissipation devices for clock generation/distribution and data serialization; wherein two PCBs are stacked up via board-to-board connectors, in which the PCBs enclose a minimum cumulative volume, wherein a ratio of PCB assembly volume to sensitive area is <15 mm with respect to a given sensitive area of the detector module; and wherein the holding means 170 for mechanically holding the components of the detector module is a thermally conductive metallic enclosure that contains and connects with the entire detector module elements and achieves thermal stability, which
i. isolates the SiPM from the PCB, further comprising a sub-unit to conductively extract the heat from the means of reading SiPMs and transferring it to the holding means to mechanically hold the components of the detector module;
ii. interfaces with a sub unit (not shown) to hold the crystal elements;
iii. houses heat sinks (not shown) and the sub unit to house the fan (not shown); and
iv. has a minimum area in the sensitive plane such that the ratio of the sensitive area to the area of the main frame is >85%.

Further, the detector module of the invention, further comprising a detector unit 110, including: at least one detector module 1101; at least one cable 1102 to deliver power, configure information and transmit serial data from the detector module to at least one interfacing electronic board, which is connected to a central data processing unit, which comprises: a de-serializer 11031, a FPGA 11032, a communication chip 11033, or a FTDI bridge chip, a clock 11016 to generate and send clock signals to the at least one detector module 1101, and a power (not shown) to provide power to the at least one detector module; and a communication hub 160 (as shown in FIG. 3) to interface with multiple interfacing electronic boards.

Further, as shown in FIG. 1(a), a PET imaging system or a SPECT according to the invention, where the system identifies a position of a patient and self-positions a detector unit 110 to scan a single or plurality of target organs sequentially along the single or a plurality of positions and orientations for each organ, comprising: a detector unit 110 to detect and capture information of gamma photons; a perception unit 140 to map a scene in the proximity of the imaging system; a robotic unit 130 to hold and position sensitive elements of the detector unit; a computer 120, or a central processing unit (CPU), interfacing with all elements of the imaging system and for controlling and processing the information; and a user interface and visualization unit 150, for accepting user instructions and displaying any selected information.

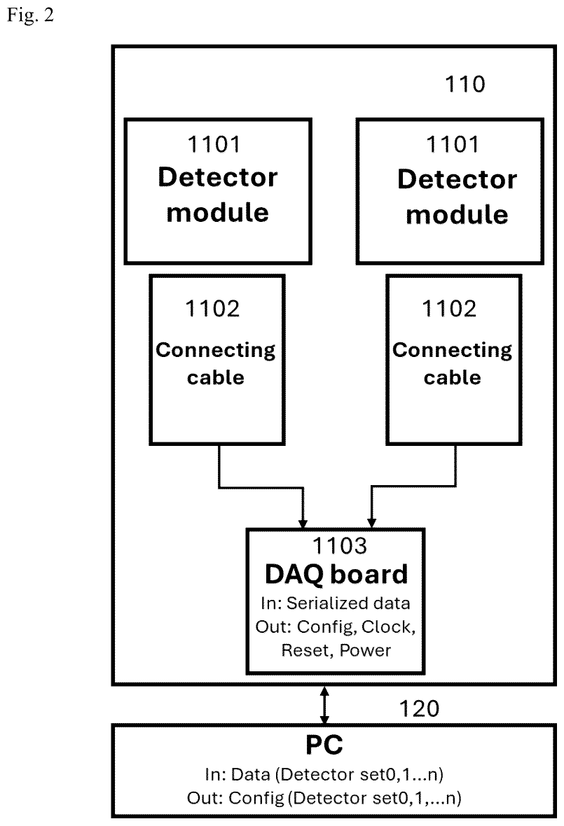

With reference to FIG. 1, while referring to FIG. 2-4, the detector unit 110 includes at least one detector module, comprising a plurality of scintillating crystals 11011 to convert gamma photons to visible light; a plurality of photodetectors 11012 to convert visible light to analog or digital electric signals; reading means 11013 for reading the analog or digital signals from each of the photodetectors to capture time of arrival and energy of a detected gamma event; at least one of the following electronic components: a configurable integrated circuit 11014, high voltage (HV) power supply with coarse and fine-adjustment, clock circuitry, and a data serializer; a plurality of PCB to house electronic components and designed to minimize a total volume of a space enclosed by the PCBs; and holding means 170 for mechanically holding the components which also acts as a means of dissipating heat within the detector module, to maximize the sensitive area while minimizing the total detector module volume.

The imaging system according to the invention, the robotic unit 130 is a six axes robotic arm, or a plural axes that achieves any possible positioning and orientation of the detector modules in a given volume, wherein the robotic unit 130 comprises a six axes robotic arm, a height adjustment arm, a distance adjustment arm to extend the reach of the six axes robotic arm, and a vertical adjustment arm for the perception unit; as shown in FIG. 1(b), wherein the robotic unit 130 further comprises a C-arm holder to hold at least one detector module at the ends of the C-arm and a connector to the rest of the robotic unit at the center of the C-arm; and wherein, as shown in FIG. 5, wherein the perception unit 140 comprises a depth sensing RGBd camera, temperature sensors for ambient temperature monitoring, and wherein the perception unit 140 tracks the patient movements and the components of the imaging system, and provides the signals to the computer to estimate and provide real time guidance to the robotic unit.

According to the invention, the computer, or the central processing unit 120, estimates from the perception unit data for the patient a precise map of the computed tomography (CT) image taken from a database of pre-existing images which can be co-registered to the given patient physically and consequently project a reconstructed PET image onto the CT image.

In the invention, the user interface and visualization unit 150 is used by a user to input settings for a current imaging plan, and to display reconstructed and co-registered images of a patient to be detected in real time.

Furthermore, the invention provides a multi-angular image reconstruction method for use in a PET system, comprising multiple iterations, each including multiple stages corresponding to different angular positions of a detector, wherein reconstruction at each stage utilizes information from other angular projections during the reconstruction, in which a fully reconstructed image that contains information from all the angular projections is achieved and referenced to a base space with a fixed coordinate axes that correspond to one of preferred angular configurations.

In the multi-angular image reconstruction method for use in the PET system of the invention, each angular orientation recovers an image from a previous angle that is reoriented to a current angle and reconstructed while accounting for sensitivity variations among different target regions due to presence of certain target regions within a field of view (FOV) of multiple angles, in which an image is rotated back to the base space at the end of each iteration, and an iterative reconstruction is performed in accordance with an algorithm as follows:

$${f}_{i}^{k + 1 , {θ}_{L - 1}} = \frac{{R}_{{θ}_{L}}^{- 1} \text{?} \{ {f}_{i}^{k + 1 - α , {θ}_{L}} \text{?} \}}{{\sum }_{jLOR} \text{?} {H}_{ij}} \times {\sum }_{jLOR} \text{?} \frac{{g}_{j}^{{θ}_{L - 1}} {H}_{ij}}{{\sum }_{{i}^{′} pixels} {R}_{θ}^{- 1} \text{?} \{ {f}_{{i}^{′}}^{k + 1 - α , {θ}_{L -}} \text{?} \} {H}_{{i}^{′} j}}$$

$$\text{?} \text{indicates text missing or illegible when filed}$$

where,
i represents a pixel number,
j represents a line of response (LOR),
k represents an iteration number from 1 to N,
L is a total number of rotation angles θ₁ . . . θ_{L},
1 represents the angular sub-iteration ranging from 0 to L−1,
α is defined such that
α=1 for 1=0 and
α=0 for 1>0,

$${R}_{{θ}_{a} , {θ}_{b}}^{- 1}$$

represents the rotation of the image from θₐ to θ_{b},
wherein for each iteration of an MLEM reconstruction, all L angular sub-iterations are performed and during each sub-iteration, an image from a previous step is rotated into a current sub-iteration and the rotated image is forward projected, and divided by the measured data for that angle, and then back projected, which result is multiplied by the ith pixel of the rotated image from the previous sub-iteration and normalized by a system response matrix, wherein each rotated orientation is reconstructed separately, then rotated to the base space and summed, in which summed image is then re-normalized to account for sensitivity variations among different regions of the target according to a number of projections in which they are a part of, wherein the PET system is a limited angle PET data system and further comprises a step by involving a number of iterations of reconstruction with each iteration further consisting a number of stages for forward image to a detector space and back detector to an image space for different projections on each separate dataset belonging to a different acquisition corresponding to a given detector positioning angle, relating to a detecting area, in which the base space is defined with coordinate axes, where the reconstructed images in each projection are rotated and integrated to achieve a complete image of the target.

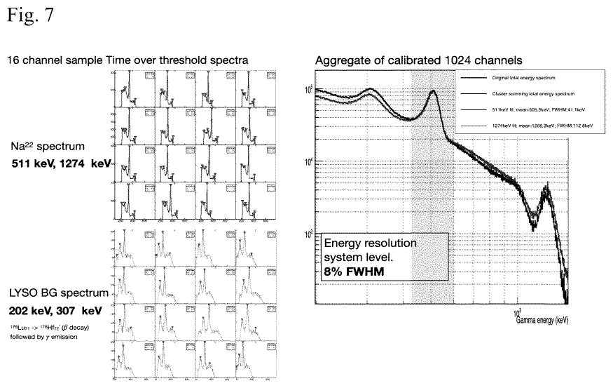

As shown in FIG. 7, it is illustrated channels threshold corrected, and calibrated for absolute gamma energy. On the left is shown a sample of 16 channels energy spectrum for a standard radioactive ²²Na source and for the intrinsic activity from the crystal. On the right is shown the energy spectrum for a standard ²²Na source for all the 1024 channels after they have been calibrated for precise energy prediction. An energy resolution of 8% FWHM was achieved.

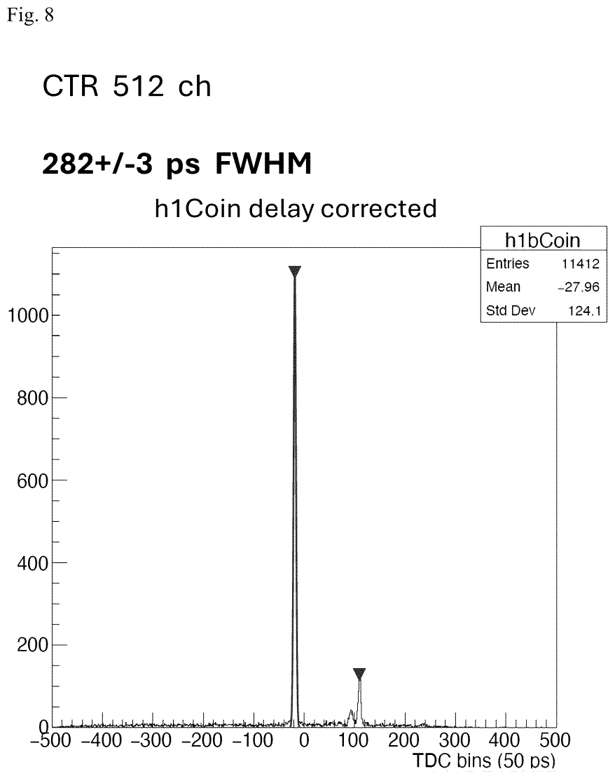

FIG. 8 shows time resolution measured as the Full Width at Half Maximum of the time delay histogram between a reference channel in opposition to all the 512 channels of a module displaying a coincidence time resolution of 282 ps FWHM.

FIG. 9 shows a breakdown of the major units of the detector unit included in the PET system of the invention, while a table 1 listing the subcomponents respectively with their reference number is presented as below, which subcomponents are held in the metal frame 170.

TABLE 1

| NO | Part name |
|---|---|
| a | Lid |
| b | Crystals |
| c | SiPM |
| d | Crystals side cover |
| e | Crystals bottom cover |
| f | PCB board |
| g | PCB connector |
| h | Connecting two board |
| i | PCB board |
| j | Connecting Fan and cover |

What is claimed is:

1. A compact, handheld gamma detector module for a positron emission tomography (PET) or single photon emission computed tomography (SPECT) imaging system, comprising:
a plurality of scintillating crystals to detect and convert gamma photons emitted by a patient to be detected to visible light;
a plurality of photodetectors to convert the visible light to analog or digital electric signals, wherein the photodetectors are silicon photo-multipliers (SiPMs);
reading means for reading the analog or digital signals from each of the photodetectors to capture time of arrival and energy of detected gamma event, wherein the reading means are application specific ICs (ASICs);
a configurable integrated circuit for configuring and reading data from the reading means, wherein the configurable integrated circuit is a field programmable gate array (FPGA) Front end;
a data serializer, for serializing the configured data, wherein the data serializer is a serializer;
a plurality of printed circuit boards (PCBs), being provided to house electronic components so as to minimize a total volume of space enclosed by the PCBs; and
holding means for mechanically holding the components, which also acts as a means of dissipating heat within the detector module, and thereby to maximize sensitive area while minimizing a total detector module volume, thereby to be unconstrained by external static structure for mechanical, power, thermal or signal flow requirements, and with minimum cabling to ease detector movement.

2. The detector module as recited in claim 1, wherein the holding means is a heat sink or a fan.

3. The detector module as recited in claim 1, wherein the scintillating crystals are coupled to the plurality of photodetectors, which are SiPM arrays, in one to one, one to many or many to one configuration maximizing light transfer.

4. The detector module as recited in claim 3, wherein each pixel of the SiPM array is connected one-to-one or many to one to the input channels of the reading means for reading photodetector signals, which is an ASIC with timing resolution value ranging from 1 picosecond to 10 nanoseconds, that digitizes the time and energy information of each gamma event and streams out the data generated in all its input channels via a high-speed interface.

5. The detector module as recited in claim 4, wherein the configurable integrated circuit is a FPGA that configures and reads data from one or several ASICs, configures the photodetectors, reads temperature sensors, and sends data to a data acquisition (DAQ) unit.

6. The detector module as recited in claim 3, wherein the gamma detecting crystals glued to the photodetector device are connected via board-to-board connectors to one side of a PCB, which contains on the other side the reading means for reading the photodetector and board-to-board connectors, where a distance from the photodetector to the reading means for reading the photodetector, to which it is connected is minimized.

7. The detector module as recited in claim 1, wherein the configurable integrated circuit is soldered to one side of a PCB, which contains on the other side board-to-board connectors and other non-high-power-dissipation devices for clock generation/distribution and data serialization.

8. The detector module as recited in claim 1, wherein two PCBs are stacked up via board-to-board connectors, in which the PCBs enclose a minimum cumulative volume, wherein a ratio of PCB assembly volume to sensitive area is <15 mm with respect to a given sensitive area of the detector module.

9. The detector module as recited in claim 3, wherein the holding means for mechanically holding the components of the detector module is a thermally conductive metallic enclosure that contains and connects with the entire detector module elements and achieves thermal stability, which
i. isolates the SiPM from the PCB, further comprising a sub-unit to conductively extract the heat from the means of reading SiPMs and transferring it to the holding means to mechanically hold the components of the detector module;
ii. interfaces with a sub unit to hold the crystal elements;
iii. houses heat sinks and the sub unit to house the fan; and
iv. has a minimum area in the sensitive plane such that the ratio of the sensitive area to the area of the main frame is >85%.

10. The detector module as recited in claim 1, further comprising a detector unit, including:
at least one detector module;
at least one cable to deliver power, configure information and transmit serial data from the detector module to at least one interfacing electronic board, which is connected to a central data processing unit, which comprises:
a de-serializer, a FPGA, a communication chip, or a FTDI bridge chip, a clock to generate and send clock signals to the at least one detector module, and a power to provide power to the at least one detector module;
and a communication hub to interface with multiple interfacing electronic boards.

11. An imaging system for use in a PET imaging system or a SPECT, where the system identifies a position of a patient and self-positions a detector unit to scan a single or plurality of target organs sequentially along the single or a plurality of positions and orientations for each organ, comprising:
a detector unit to detect and capture information of gamma photons;
a perception unit to map a scene in the proximity of the imaging system;
a robotic unit to hold and position sensitive elements of the detector unit;
a computer, or a central processing unit (CPU), interfacing with all elements of the imaging system and for controlling and processing the information; and
a user interface and visualization unit, for accepting user instructions and displaying any selected information.

12. The imaging system as recited in claim 11, wherein the detector unit includes at least one detector module, comprising
a plurality of scintillating crystals to convert gamma photons to visible light;
a plurality of photodetectors to convert visible light to analog or digital electric signals;
reading means for reading the analog or digital signals from each of the photodetectors to capture time of arrival and energy of a detected gamma event;
at least one of the following electronic components: a configurable integrated circuit, high voltage (HV) power supply with coarse and fine-adjustment, clock circuitry, and a data serializer;
a plurality of PCB to house electronic components and designed to minimize a total volume of a space enclosed by the PCBs; and
holding means for mechanically holding the components which also acts as a means of dissipating heat within the detector module, to maximize the sensitive area while minimizing the total detector module volume.

13. The imaging system as recited in claim 11, wherein the robotic unit is a six axes robotic arm that achieves any possible positioning and orientation of the detector modules in a given volume.

14. The imaging system as recited in claim 11, wherein the robotic unit comprises a six axes robotic arm or a plural axes, a height adjustment arm, a distance adjustment arm to extend the reach of the six axes robotic arm, and a vertical adjustment arm for the perception unit.

15. The imaging system as recited in claim 11, wherein the robotic unit further comprises a C-arm holder to hold at least one detector module at the ends of the C-arm and a connector to the rest of the robotic unit at the center of the C-arm.

16. The imaging system as recited in claim 11, wherein the perception unit comprises a depth sensing RGBd camera, temperature sensors for ambient temperature monitoring.

17. The imaging system as recited in claim 11, wherein the perception unit tracks the patient movements and the components of the imaging system, and provides the signals to the computer to estimate and provide real time guidance to the robotic unit.

18. The imaging system as recited in claim 11, wherein the computer, or the central processing unit estimates from the perception unit data for the patient a precise map of the computed tomography (CT) image taken from a database of pre-existing images which can be co-registered to the given patient physically and consequently project a reconstructed PET image onto the CT image.

19. The imaging system as recited in claim 11, wherein the user interface and visualization unit is used by a user to input settings for a current imaging plan, and to display reconstructed and co-registered images of a patient to be detected in real time.

20. A multi-angular image reconstruction method for use in a PET system, comprising multiple iterations, each including multiple stages corresponding to different angular positions of a detector, wherein reconstruction at each stage utilizes information from other angular projections during the reconstruction, in which a fully reconstructed image that contains information from all the angular projections is achieved and referenced to a base space with a fixed coordinate axes that correspond to one of preferred angular configurations.

21. The multi-angular image reconstruction method for use in the PET system as claimed in claim 20, wherein each angular orientation recovers an image from a previous angle that is reoriented to a current angle and reconstructed while accounting for sensitivity variations among different target regions due to presence of certain target regions within a field of view (FOV) of multiple angles, in which an image is rotated back to the base space at the end of each iteration, and an iterative reconstruction is performed in accordance with an algorithm as follows:
$${f}_{i}^{k + 1 , {θ}_{L - 1}} = \frac{{R}_{{θ}_{L}}^{- 1} \text{?} \{ {f}_{i}^{k + 1 - α , {θ}_{L}} \text{?} \}}{{\sum }_{jLOR} \text{?} {H}_{ij}} \times {\sum }_{jLOR} \text{?} \frac{{g}_{j}^{{θ}_{L - 1}} {H}_{ij}}{{\sum }_{{i}^{′} pixels} {R}_{θ}^{- 1} \text{?} \{ {f}_{{i}^{′}}^{k + 1 - α , {θ}_{L -}} \text{?} \} {H}_{{i}^{′} j}}$$
$$\text{?} \text{indicates text missing or illegible when filed}$$
where,
i represents a pixel number,
j represents a line of response (LOR),
k represents an iteration number from 1 to N,
L is a total number of rotation angles θ₁ . . . θ_{L},
1 represents the angular sub-iteration ranging from 0 to L−1,
α is defined such that
α=1 for 1-0 and
α=0 for 1>0,
$${R}_{{θ}_{a} , {θ}_{b}}^{- 1}$$
represents the rotation of the image from θₐ to θ_{b},
wherein for each iteration of an MLEM reconstruction, all L angular sub-iterations are performed and during each sub-iteration, an image from a previous step is rotated into a current sub-iteration and the rotated image is forward projected, and divided by the measured data for that angle, and then back projected, which result is multiplied by the ith pixel of the rotated image from the previous sub-iteration and normalized by a system response matrix,
wherein each rotated orientation is reconstructed separately, then rotated to the base space and summed, in which summed image is then re-normalized to account for sensitivity variations among different regions of the target according to a number of projections in which they are a part of.

22. The multi-angular image reconstruction method for use in the PET system as claimed in claim 21, wherein the PET system is a limited angle PET data system and further comprises a step by involving a number of iterations of reconstruction with each iteration further consisting a number of stages for forward image to a detector space and back detector to an image space for different projections on each separate dataset belonging to a different acquisition corresponding to a given detector positioning angle, relating to a detecting area, in which the base space is defined with coordinate axes, where the reconstructed images in each projection are rotated and integrated to achieve a complete image of the target.
